(19) United States
(12) Patent Application Publication
(10) Pub. No.: US 2026/0364058 A1
(43) Pub. Date: Nov. 19, 2026

(54) ANTIBIOTIC COMBINATION THERAPIES

(71) Applicant: BIOVERSYS AG, Basel (CH)

(72) Inventors: Sergio LOCIURO, Basel (CH); Glenn E. DALE, Basel (CH); Marc GITZINGER, Laufenburg (CH); Vincent TREBOSC, Rixheim (FR)

(73) Assignee: BIOVERSYS AG, Basel (CH)

(21) Appl. No.: 19/752,900

(22) Filed: Jul. 24, 2026

Related U.S. Application Data
(63) Continuation of application No. 17/978,647, filed on Nov. 1, 2022, now abandoned.
(60) Provisional application No. 63/342,217, filed on May 16, 2022.

(51) Int. Cl. A61K 31/438; A61K 38/12; A61P 31/04; C07K 7/62

(52) U.S. Cl. CPC A61K 31/438; A61K 38/12; A61P 31/04; C07K 7/62

(57) ABSTRACT

The invention provides antibiotic combination therapies for treating an A. baumannii infection in a subject. The combination therapies include rifabutin and a member of the polymyxin class of antibiotics, such as commercially available natural products antibiotics, e.g., polymyxins B and polymyxins E (colistin and its pro-drug colistin methane sulfonate (CMS)) and synthetic or semi-synthetic derivatives and analogs, e.g., SPR206, MRX-8 and QPX9003 and polymyxin-like antibiotics, e.g., Pol7306

### CROSS-REFERENCE TO RELATED APPLICATIONS

This Application claims the benefit of, and priority to, U.S. Provisional Application No. 63/342,217, filed May 16, 2022, the content of which is hereby incorporated by reference in its entirety.

### FIELD OF THE INVENTION

The invention relates generally to rifabutin combination therapies for treating A. baumannii infections.

### BACKGROUND

The emergence of multi-drug resistant (MDR) or extensively-drug resistant (XDR) strains of bacteria over the last few decades has made bacterial infections an increasingly serious public health concern. One bacterial species that poses a major health threat is Acinetobacter baumannii, which can cause pneumonia, meningitis, and infections of the blood, urinary tract, and skin. Because A. baumannii cells can survive on artificial surfaces for extended periods, the bacterium is readily transmissible in a hospital environment, and most A. baumannii infections are nosocomially acquired. For example, many soldiers in the Middle East have been infected with A. baumannii while being treated for injuries sustained during combat, and multidrug-resistant strains of the bacterium represent a significant complication in rehabilitation of injured soldiers.

Treatment of A. baumannii infections is challenging. Through the use of multiple strategies, strains of A. baumannii have developed resistance to antibiotics in several different classes, including aminoglycosides, aminocyclitols, tetracyclines, chloramphenicol, and carbapenems. Polymyxins, such as colistin, are typically used as a last resort due to their serious side effects, but some A. baumannii strains are resistant to colistin as well (Zubair et al, 2015, Colistin-resistant Acinetobacter baumannii: beyond carbapenem resistance, Clin Infect Dis. 60(9): 1295-303; Trebosc et al, Dissecting Colistin Resistance Mechanisms in Extensively Drug-Resistant Acinetobacter baumannii Clinical Isolates, Therapeutics and Prevention, July/August 2019 Volume 10 Issue 4 e01083-19; the content of each of which is incorporated herein by reference). Consequently, current tools for treating and preventing illness caused by this bacterium are inadequate for many patients. Significant efforts have been made to find out a solution in order to treat theses nosocomial pathogen, one of which is combined therapy (Levin et al, 1999; Wood et al, 2003). The combinations of two antibiotics have shown different effects on each other and in many cases the effect is synergistic or strengthening but, in some cases, antagonism is observed (Montero et al, 2004; Tripodi et al, 2007). The unexpected activity of rifabutin, an antibiotic belonging to the rifamycin class, against A. baumannii has been recently reported (US20210077471A1, the content of which is incorporated herein by reference in its entirety). Also, synergy of rifabutin with the polymyxin class of antibiotics, a class which includes commercially available natural products antibiotics such as polymyxins B and polymyxins E (colistin and its pro-drug colistin methane sulfonate (CMS)) and synthetic or semi-synthetic derivatives and analogs currently in clinical development such as SPR206, MRX-8 (Vaara, 2019, Polymyxins and Their Potential Next Generation as Therapeutic Antibiotics, Front. Microbiol. 2019; 10:1689, the content of which is incorporated herein by reference in its entirety) and QPX9003 (Roberts et al, 2019, the content of which is incorporated herein by reference in its entirety), has been recently reported (US20210077472A1, the content of which is incorporated herein by reference in its entirety).

### SUMMARY

The invention provides combination therapies that include rifabutin and a polymyxin (e.g., colistin, colistin methane sulfonate (CMS), polymyxin B, polymyxin derivatives and analogues as exemplified by SPR 206, MRX-8 and QPX 9003) for treating A. baumannii infections. The invention is based on the finding that rifabutin acts synergistically with these antibiotics to inhibit growth of A. baumannii cells such that the individual doses of each drug in the combination therapy may be below the therapeutic dose if given alone. By allowing for lower doses of drugs with potential toxicity at their standard therapeutic doses (e.g., a polymyxin), they can be used in more cases and in earlier interventions providing another tool in combating A. baumannii infections where drug resistance is a significant concern.

The combinations of antibiotics described herein display synergy to a wide range of A. baumannii strains. The synergy greatly increases the susceptibility of A. baumannii cells to rifabutin and a polymyxin, with MIC90 (the maximum Minimal Inhibitory Concentration which inhibits growth of 90% of the strains of a test panel) of rifabutin being up to 32 fold lower when in the presence of subinhibitory concentrations/subtherapeutic exposures of a polymyxin antibiotic. Unexpectedly, the presence of subinhibitory concentrations/subtherapeutic exposures of a polymyxin antibiotic made rifabutin more rapidly bactericidal and prevented regrowth (prevented the selection of resistant colonies) in time-kill experiments.

Aspects of the invention may include methods for treating an A. baumannii infection in a subject where the method may comprise providing to a subject infected with A. baumannii a therapeutic or subtherapeutic dose of rifabutin and a subtherapeutic dose of a polymyxin. The therapeutic dose of rifabutin being described as a dose able to achieve a Cₘₐₓ of about 2 mg/L and a 24-hour exposure (AUC)>10 mg*h/L. The subtherapeutic dose of rifabutin may be defined as any dose determining a Cₘₐₓ<2 mg/L and a 24-hour AUC≤10 mg*h/L. The subtherapeutic dose of intravenously administered rifabutin may be about two thirds of a standard therapeutic dose or less.

In certain embodiments, a subtherapeutic dose of a polymyxin antibiotic is defined as a dose in which the mean fCₘₐₓ, fAUC and fC_{trough} required for clinical efficacy if given alone is not achieved and the fC_{trough} of that polymyxin is comprised between its MICs and its MIC50 (i.e. the concentration at which growth of 5 to 50% of the strains in a test panel is inhibited). In certain embodiments, the subtherapeutic dose of a polymyxin is that required for achieving a fC_{trough} that is one half the fC_{trough} of the therapeutic dose or less. In certain embodiments, the subtherapeutic dose of a polymyxin is that required for achieving a fC_{trough} that is one quarter the fC_{trough} of the therapeutic dose or less. In some embodiments, the therapeutic dose of intravenous rifabutin can be about 600 mg/day. In some embodiments, the subtherapeutic dose of intravenous rifabutin can be about 550 mg/day or less. The subtherapeutic dose of intravenous rifabutin may be about 400 mg/day or less. In various embodiments, the subtherapeutic dose of intravenous rifabutin can be between about 250 mg/day and about 400 mg/day.

The rifabutin and the polymyxin may be provided in a single formulation suitable for intravenous administration, wherein the polymyxin may be polymyxin B and the subtherapeutic dose of the polymyxin can be about 60 mg/day or less or the polymyxin may be colistin methane sulfonate (CMS) and the subtherapeutic dose of the polymyxin can be about 90 mg/day or less.

In certain embodiments, the rifabutin and the polymyxin can be provided separately, wherein the polymyxin may be polymyxin B and the subtherapeutic dose of the polymyxin may be between about 0.5 mg/kg/day and about 0.8 mg/kg/day or the polymyxin may be colistin methane sulfonate (CMS) and the subtherapeutic dose of the polymyxin can be between about 0.8 mg/kg/day and about 1.6 mg/kg/day.

In various embodiments, the rifabutin may be administered intravenously or orally or by inhalation. The A. baumannii may comprise carbapenem-resistant A. baumannii (CRAB). The polymyxin can be SPR206 or QPX9003 or MRX-8 or any other synthetic or semi-synthetic polymyxin or polymyxin-like antibiotic (e.g. Pol7306) and the subtherapeutic dose of the polymyxin is about one third of a standard therapeutic dose.

Aspects of the invention may include combination therapies comprising rifabutin and a polymyxin in a therapeutically effective amount to treat an A. baumannii infection in a subject, wherein the rifabutin and the polymyxin are present in amounts that would be subtherapeutic if provided alone.

### BRIEF DESCRIPTION OF THE DRAWINGS

FIG. 1 shows cumulative index and MIC50 and MIC90 of rifabutin in the presence of members of the polymyxin family of antibiotics at sub MIC (sub fC_{trough} concentrations).

FIG. 2 shows the MIC distribution of rifabutin in the presence of members of the polymyxin family of antibiotics at sub MIC (sub fC_{trough} concentrations).

FIG. 3 shows kill curve results on a first A. baumannii strain.

FIG. 4 shows kill curve results on a second A. baumannii strain.

FIG. 5 shows time kill curves of low concentrations of rifabutin at very low concentration and in the presence of polymyxin B at sub MIC (sub free trough concentrations) on A. baumannii Pop1 strains.

FIG. 6 shows time kill curves of rifabutin at very low concentration and in the presence of polymyxin B at sub MIC (sub free trough concentrations) on A. baumannii Pop2 strains.

### DETAILED DESCRIPTION

The invention provides combination therapies for treating an A. baumannii infection in a subject. The combination therapies are based on the finding that rifabutin acts synergistically with polymyxin antibiotics such as polymyxin B and polymyxin E (colistin) and synthetic or semi-synthetic derivatives and analogs currently in clinical development such as SPR206, MRX-8 and QPX9003 to inhibit growth of A. baumannii cells even at what would be subtherapeutic doses of the individual antibiotics if given alone. Therefore, while polymyxins like colistin are antibiotics of last resort due to potential toxicity and adverse events, the ability to reduce the dose when used in combination with rifabutin may open up new applications providing another tool in combating increasingly resistant infections.

The activity of rifabutin against A. baumannii, including multi-drug resistant (MDR) and carbapenem-resistant A. baumannii (CRAB) has been recently reported in US20210077471A1 along with an intravenous dosage form of rifabutin in US20210077470A1, the content of each of which is incorporated herein in its entirety. Additionally, a synergistic effect between rifabutin and the polymyxin class of antibiotics has been reported in US20210077472A1, incorporated herein by reference in its entirety.

A surprising benefit of this synergistic effect is that a low dose of a member of the polymyxin class of antibiotics (as low as ¼ of the normally prescribed dose) together with a low dose rifabutin (as low as ¼ of the effective dose) of the required dose to achieve a Cₘₐₓ of about 2 mg/L and/or AUC of >10 mg*h/L and <300 mg*h/L) can be administered to patients affected by A. baumannii infections such as CRAB.

Accordingly, methods and formulations of the invention comprise combination therapies of rifabutin at therapeutic or subtherapeutic doses and a polymyxin at doses that would be considered subtherapeutic. In various embodiments, rifabutin can be administered together with the member of the polymyxin class of antibiotics in a single vial (co-formulation) for reconstitution and dilution in the proper diluent for infusion to humans. Alternatively, rifabutin and the member of the polymyxin class of antibiotics can be contained in different vials to be independently reconstituted and combined in the proper diluent at the moment of infusion to humans. In still other embodiments, rifabutin and the member of the polymyxin class of antibiotics can be administered separately to patients, either consequently in the same infusion location or in parallel in two different infusion locations. Rifabutin can also be administered orally and the member of the polymyxin class of antibiotics can be administered by infusion or any other appropriate route of administration (e.g., inhalation). In fact, rifabutin and the member of the polymyxin class of antibiotics can be administered together or separately both by inhalation.

Therapeutic doses of intravenous rifabutin are about 300 mg BID (i.e. 600 mg/day). When used in combination with a polymyxin according to methods of the invention, the amount of rifabutin administered intravenously (IV) in the combination therapy may be less than about 600 mg/day (e.g., subtherapeutic if given alone). In some embodiments, the IV “subtherapeutic” rifabutin dose may be more than about 150 mg/day and, in preferred embodiments, between about 250 and about 500 mg/day (125-250 mg BID).

Standard therapeutic polymyxin dosing is discussed in Tsuji B T, Pogue J M, Zavascki A P, et al. International Consensus Guidelines for the Optimal Use of the Polymyxins: Endorsed by the America College of Clinical Pharmacology (ACCP), European Society of Clinical Microbiology and Infectious Diseases (ESCMID), Infectious Diseases Society of America (IDSA), International Society for Anti-infective Pharmacology (ISAP), Society of Critical Care Medicine (SCCM), and Society of Infectious Diseases Pharmacists (SIDP). Pharmacotherapy 2019; 39 (1): 10-39, incorporated herein by reference in its entirety.

Standard therapeutic doses of polymyxin B may be between about 1.5 and about 2.5 mg/kg/day. If administered separately from rifabutin, polymyxin B may be dosed by mg/kg and may be between about 0.5 and about 0.8 mg/kg/day. If co-formulated with rifabutin, polymyxin B can be provided in a fixed dose, for example at about 60 mg/day (30 mg BID).

Standard therapeutic doses of CMS may be between about 2.5 and about 5.0 mg/kg/day. If administered separately from rifabutin, CMS may be dosed by mg/kg and may be between about 0.8 and about 1.6 mg/kg/day. If co-formulated with rifabutin, CMS can be provided in a fixed dose, for example at about 90 mg/day (45 mg BID).

Genearlly, a subtherapeutic dose of a polymyxin antibiotic is considered a dose in which the mean fCₘₐₓ, fAUC and fC_{trough} required for efficacy if given alone is not achieved and/or where the fC_{trough} of that polymyxin is between its MIC₅ and its MIC₅₀ (i.e. the concentration at which growth of 5 to 50% of the strains in a test panel of more than 100 recent A. baumannii clinical isolates is inhibited). fCₘₐₓ should be understood to be the pharmacokinetic maximum free-drug concentration achieved by a drug at a given dose at steady state. fC_{trough} should be understood to be the pharmacokinetic minimum free-drug concentration achieved by a drug in between doses at steady state at a given dose. fAUC should be understood to be the pharmacokinetic free-drug Area Under the Curve (i.e., the total exposure-generally in 24 hours) achieved by a drug at a given dose at steady state.

### Combination Therapies

The combination therapies of the invention include two antibiotics that act synergistically to inhibit growth of A. baumannii cells. Synergy between antibiotics, such as a rifabutin and polymyxin B or colistin or a synthetic or semi-synthetic derivative or analog or polymyxin-like antibiotics in clinical development such as SPR206, MRX-8, QPX9003, or Pol7306, may be determined by any suitable method.

The combination therapies of the invention, one of the antibiotics is rifabutin. The combination therapies include a polymyxin, such as polymyxin B or colistin or a synthetic or semi-synthetic derivative or analog or polymyxin-like antibiotics in clinical development such as SPR206, MRX-8, QPX9003, or Pol7306 that acts synergistically with the rifabutin. In the clinic, colistin may be provided as colistimethate sodium or colistin sulfate. In case of polymicrobial infections, the inclusion of any additional antibiotics to the combination therapy are contemplated herein and would be apparent to one of ordinary skill in the art and may include 3, 4, 5, or more different antibiotics. Exemplary additional antibiotics may include aminocyclitol, aminoglycoside, beta-lactam, beta-lactamase inhibitor, carbapenem, cephalosporin, quinolone, rifamycin, sulfonamide, minocycline, eravacycline, sulbactam, or tetracycline. Each antibiotic may independently be amikacin, trimethoprim-sulfamethoxazole, cefepime, cefiderocol, ceftazidime, chloramphenicol, ciprofloxacin, colistin, doripenem, gentamicin, imipenem, levofloxacin, meropenem, penicillin, piperacillin, polymyxin B, rifampicin, tazobactam, or tigecycline.

Any additional antibiotic may be administered by any suitable route of administration. For example, and without limitation, each antibiotic may independently be administered intravenously, orally, parenterally, subcutaneously, by inhalation, by injection, and/or by infusion.

Additional antibiotics may be administered to the same dosing regimen. One or more antibiotics may be administered according to different dosing regimens. A dosing regimen may include a dosage, a schedule or administration, or both. A dosage may be described by an absolute amount of drug (e.g. mg), or by a relative amount of the drug to the subject (e.g. mg/kg). A schedule of administration may be described by the interval between doses. For example and without limitation, the interval between doses may be about an hour, about 2 hours, about 3 hours, about 4 hours, about 6 hours, about 8 hours, about 12 hours, about 24 hours, about 36 hours, about 48 hours, about 3 days, about 4 days, about 5 days, about 6 days, about 7 days, or more.

### Formulations

One or more of the antibiotics may be provided in a single formulation. One or more antibiotics may be provided in separate formulations. Each formulation may be prepared for delivery by a particular route of administration, such as intravenously, orally, parenterally, subcutaneously, by inhalation, by injection, and/or by infusion.

The antibiotics may be provided as pharmaceutically acceptable salts, such as nontoxic acid addition salts, which are salts of an amino group formed with inorganic acids such as but not limited to hydrochloric acid, hydrobromic acid, phosphoric acid, sulfuric acid and perchloric acid or with organic acids such as, but not limited to, acetic acid, maleic acid, tartaric acid, citric acid, succinic acid, methansulfonic acid, glucuronic acid, malic acid, gluconic acid, lactic acid, aspartic acid, or malonic acid.

The formulation may be administered by injection, infusion, implantation (intravenous, intramuscular, subcutaneous, or the like) or by inhalation in dosage forms, formulations, or via suitable delivery devices or implants containing conventional, non-toxic pharmaceutically acceptable carriers, solvents, diluents, and adjuvants. The formulation and preparation of such compositions are well known to those skilled in the art of pharmaceutical formulation.

Formulations for parenteral use may be provided in unit dosage forms (e.g., in single-dose ampoules and vials), in vials containing several doses and in which a suitable preservative may be added (see below), in prefilled syringes, or in prefilled IV bags. The pharmaceutical compositions described herein may be in the form suitable for sterile injection.

Formulations may include solutions containing rifabutin. Rifabutin solutions and methods of making rifabutin solutions are described in US20210077470A1, the contents of which are incorporated herein by reference in its entirety.

Depending upon the needs of the patient, and the clinical conditions, administration of the composition by IV administration may be favored over oral administration because it allows for rapid introduction of the antibiotic into systemic circulation, provides complete bioavailability, allows to better control the pharmacokinetic parameters that are driving the pharmacological efficacy, and avoids issues of stability in the gastrointestinal tract and absorption.

### Treating a. Baumannii Infections

The combination therapies of the invention are useful for treating an A. baumannii infection in a subject. The subject may be a human. The subject may be a pediatric, a newborn, a neonate, an infant, a child, an adolescent, a pre-teen, a teenager, an adult, or an elderly subject. The subject may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting.

The subject may have an A. baumannii infection that is resistant to an antibiotic or to multiple classes of commercially available antibiotics, e.g., MDR, XDR or PDR A. baumannii infections. Importantly, the combination therapies of the invention are useful for treating infections caused by A. baumannii strains with elevated MIC towards rifabutin and/or resistance to polymyxin B or colistin or a synthetic or semi-synthetic derivative or analog in clinical development such as SPR206, MRX-8, QPX9003, or Pol7306.

The antibiotics in the combination therapy of this invention may be administered simultaneously or sequentially. Sequential administration or alternating administration may include providing each antibiotic exclusively for a period of time. Sequential administration may include a period of overlap in which the subject is provided both the formulation containing rifabutin and the formulation containing the polymyxin.

### EXAMPLES

### Example 1

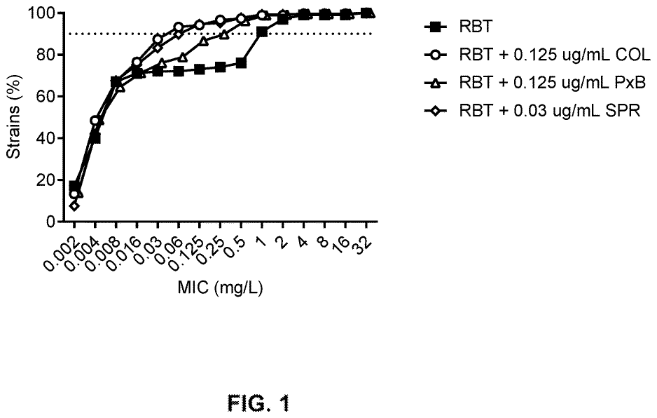

The MIC₉₀ (the maximum Minimal Inhibitory Concentration which inhibits growth of 90% of the strains of a test panel) of rifabutin is 2 or 1 mg/L when tested respectively in RPMI/FCS for broth microdilution or MHA/PIH for agar dilution against a panel of 293 carbapenem-resistant Acinetobacter baumannii (CRAB) (Trebosc V, et al. 2020. In vitro activity of rifabutin against 293 contemporary carbapenem-resistant Acinetobacter baumannii clinical isolates and characterization of rifabutin mode of action and resistance mechanisms. J Antimicrob Chemother. 75(12):3552-3562, incorporated herein by reference). When tested in CAMHB/PIH by broth microdilution in the presence of about one-quarter of the fC_{trough} (free drug concentration at trough) of various members of the polymyxin class of antibiotics the MIC₉₀ of rifabutin dropped significantly to 0.06-0.25 mg/L in the presence 0.125 mg/L of polymyxin B, 0.125 mg/L of colistin, and 0.03 mg/L of SPR 206. At these respective polymyxin concentrations, no strains, 3% of the strains, and <5% of the strains would be susceptible to the respective polymyxin. FIG. 1 illustrates the cumulative index and MIC₅₀ and MIC₉₀ of rifabutin in the presence of members of the polymyxin family of antibiotics at sub MIC (sub trough concentrations) showing the synergistic effect discussed above.

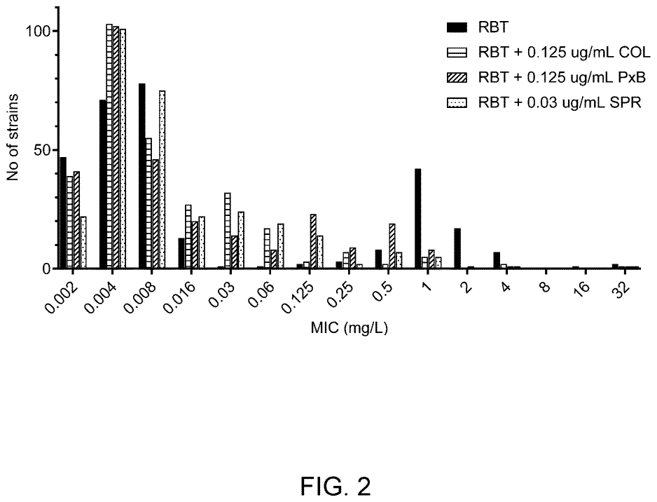

In addition, the rifabutin MIC distribution presented three distinct sub-population when tested in MHA/PIH, with population 1 (MICs<0.125 mg/L) representing 72% of the strains, population 2 (MICs≥0.125 to <16 mg/L) representing 27% of the strains and population 3(MICs ≥16 mg/L) representing 1% of the strains) (Trebosc V, et al. 2020. In vitro activity of rifabutin against 293 contemporary carbapenem-resistant Acinetobacter baumannii clinical isolates and characterization of rifabutin mode of action and resistance mechanisms. J Antimicrob Chemother. 75(12):3552-3562, incorporated herein by reference in its entirety). When tested in CAMHB/PIH by broth microdilution in the presence of about one-quarter of the fC_{trough} (free drug concentration at trough) of various members of the polymyxin class of antibiotics the MIC distributin of rifabutin presented only one main strain population with 99% of the strains having an MIC≤1 mg/L. This indicates that the combination is active on strains with elevated rifabutin MIC. Importantly, 45, 40 and 18 strains had an MIC>2 mg/L towards colistin, polymyxin B and SPR206, respectively, indicating that the combination is active on strains resistant to a polymyxin. Together, these results suggest that the combination is active on strains with elevated rifabutin MIC, strains resistant to a polymyxin, or both. FIG. 2 illustrates the MIC distribution of rifabutin in the presence of members of the polymyxin family of antibiotics at sub MIC (sub trough concentrations) showing the synergistic effect discussed above.

### Example 2

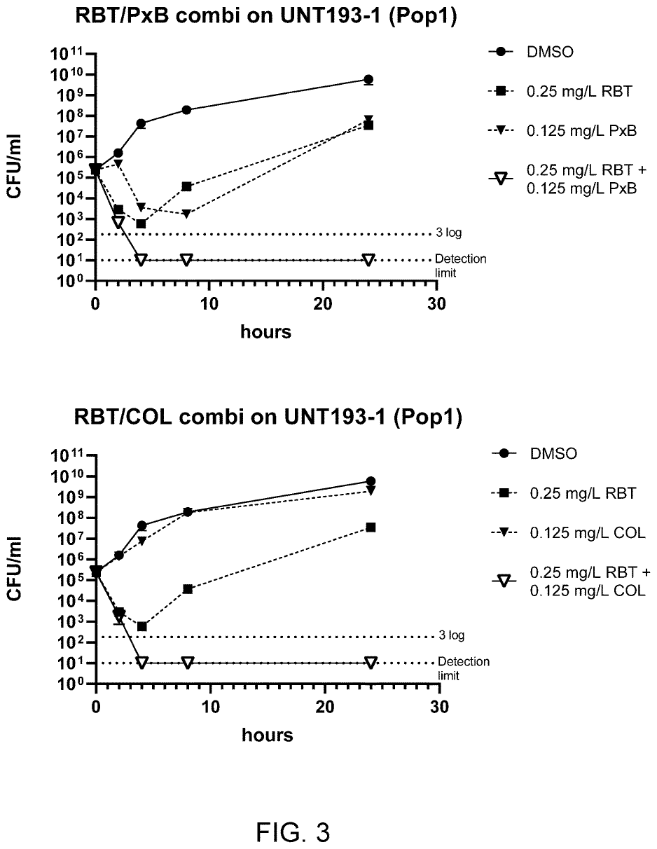

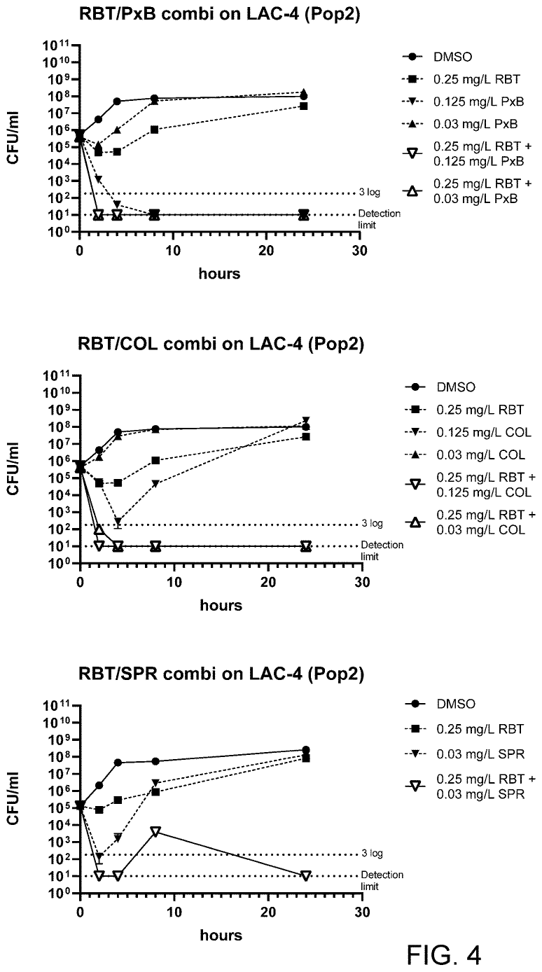

Based on the data of EXAMPLE 1, the bactericidal activity and the resistance development of rifabutin and of fixed rifabutin/polymyxin concentrations was assessed in time-kill curve experiments. Concentrations were fixed at 0.25 mg/L for rifabutin (averagely one eighth the rifabutin MIC₉₀), at 0.125 mg/L and 0.03 mg/L for colistin and polymyxin B respectively (one fourth the fC_{trough} of these polymyxins or lower) and at 0.03 mg/L for SPR 206 (a concentration at which growth of less than 5% of the strains is inhibited if given alone). Low concentration rifabutin+low concentration of a member of the polymyxin class of antibiotics was at least as bactericidal as a full dose rifabutin or a full dose of a polymyxin antibiotic and was fully preventive of resistance development as shown in FIGS. 3 and 4. FIGS. 3 and 4 show kill curve experiments on two different A. baumannii strains (Pop1 and Pop2) having very different rifabutin MICs. As shown, rifabutin at 0.25 mg/L as well as colistin or polymyxin B at 0.125 or 0.03 mg/L or SPR 206 at 0.03 mg/L are either poorly bactericidal or allow for regrowth in both populations. However, the combination of rifabutin with one of the polymyxin, provided together at the low dosages, is highly and rapidly bactericidal, indicating that the combination is synergistic and protects from resistance development (bacterial regrowth).

FIG. 3 shows time kill curves of rifabutin in the presence of members of the polymyxin family of antibiotics at sub MIC (sub trough concentrations) on an A. baumannii Pop1 strain (rifabutin MIC=0.016 mg/L).

FIG. 4 shows time kill curves of rifabutin in the presence of members of the polymyxin family of antibiotics at sub MIC (sub trough concentrations) on an A. baumannii Pop2 strain (rifabutin MIC=1 mg/L).

### Example 3

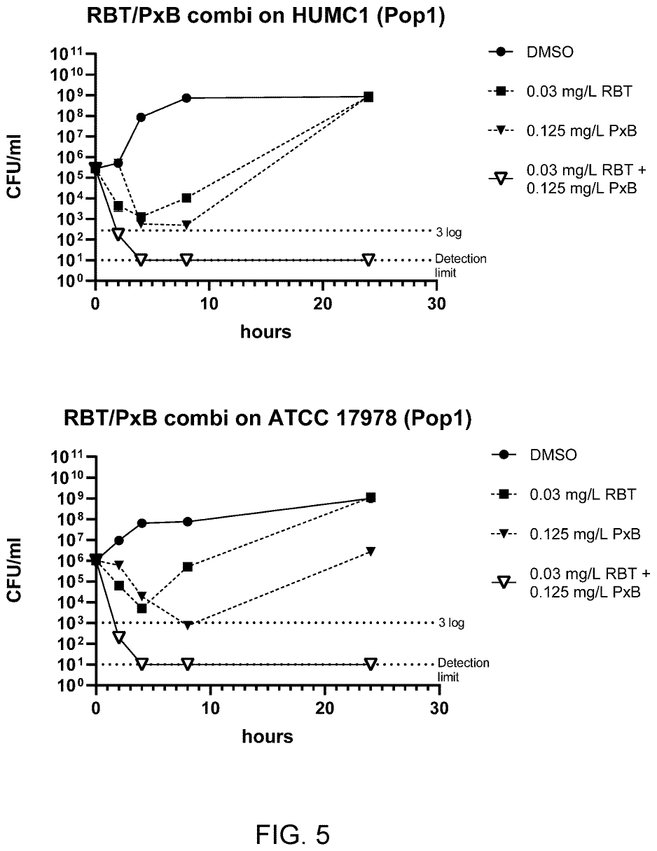

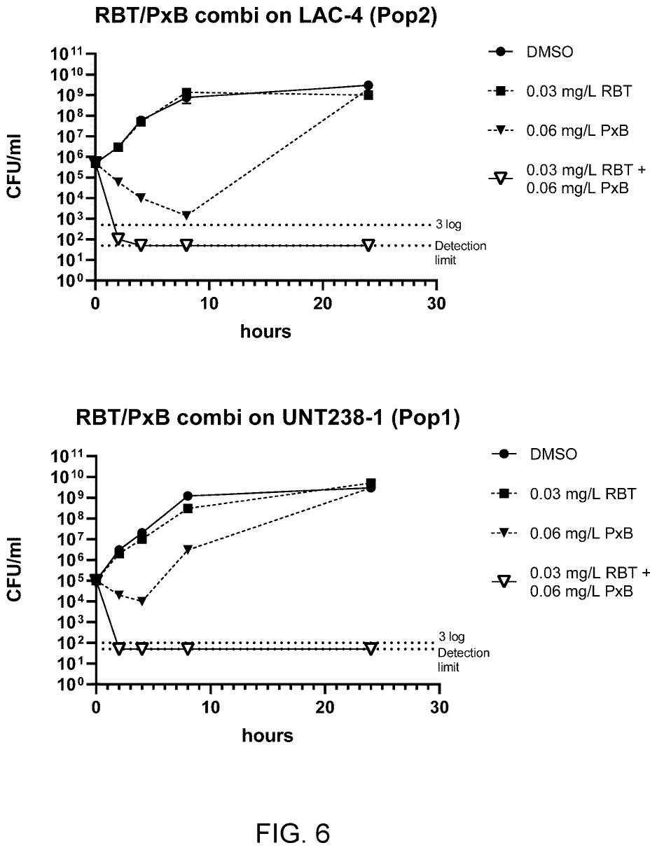

Based on the data of EXAMPLE 2, the bactericidal activity and the resistance development of rifabutin/polymyxin B was assessed in time kill curve experiments using a lower concentration of rifabutin and additional strains. Concentrations were fixed at 0.03 mg/L for rifabutin (on average, one thirty-second the rifabutin MIC₉₀), and at 0.125 or 0.06 mg/L for polymyxin B (one fourth and one eighth the fC_{trough} of polymyxin B). That very low concentration of rifabutin along with a low concentration of polymyxin B was at least as bactericidal as a full dose of rifabutin or a full dose of polymyxin B and was fully preventive of resistance development as shown in FIGS. 5 and 6. FIG. 5 shows kill curve experiments on two A. baumannii strains with low rifabutin MICs (Pop1) and FIG. 6 shows kill curve experiments on two A. baumannii strains with higher rifabutin MICs (Pop2). As shown, rifabutin at 0.03 mg/L as well as polymyxin B at 0.125 or 0.06 mg/L were either poorly bactericidal or allowed for regrowth in both populations. However, the combination of rifabutin with polymyxin B, provided together at those low dosages, is highly and rapidly bactericidal, indicating that the combination is synergistic and protects from resistance development (bacterial regrowth) even at very low concentrations of rifabutin.

FIG. 5 shows time kill curves of rifabutin at very low concentration and in the presence of polymyxin B at sub MIC (sub free trough concentrations) on A. baumannii Pop1 strains (rifabutin MIC=0.002 mg/L and 0.008 mg/L for HUMC1 and ATCC 17978, respectively).

FIG. 6 shows time kill curves of rifabutin at very low concentration and in the presence of polymyxin B at sub MIC (sub free trough concentrations) on A. baumannii Pop2 strains (rifabutin MIC=1 mg/L and 2 mg/L for LAC-4 and UNT238-1, respectively).

### Methods

### Antibacterial Agents

Rifabutin (batch no. BV-015-3219-001-03) was manufactured by Olon S.p.A. and 10 g/L stock solutions were prepared in DMSO. Stock solutions of rifampicin (Sigma R3501) were prepared at 10 mg/mL in DMSO. Stock solutions of colistin sulfate (Sigma C4461), polymyxin B sulfate (Sigma P4932) and SPR206 acetate (MedChemExpress, HY-128780B) were prepared at 10 mg/mL in water. Stock solutions were stored at −20 ° C. until use.

### Bacterial Strains

The A. baumannii clinical isolates used in this study are from the BioVersys strain collection. The strains were stored at −80 ° C. as 20% (v/v) glycerol stock cultures.

### Antimicrobial Susceptibility And Synergy Testing

The rifabutin MICs in combination with a fixed concentration of the specified polymyxin were determined by broth microdilution method according to the CLSI guideline. The medium used to determine the MICs was cation-adjusted Muller Hinton broth (CAMHB) supplemented with 0.1 mM of pyridoxal isonicotinoyl hydrazone (PIH). As previously described in Trebosc et al. 2020, the rifabutin MIC in this medium may be affected by the skipped well phenomenom, where wells growing in concentrations above the concentration of the first inhibited well do not reflect the proper MIC of the strain. Accordingly, the MIC values were set at the first lowest rifabutin concentration preventing growth as assessed by visual inspection of the assay plate.

### Time Kill Curves

Time-kill kinetics were performed in CAMHB supplemented with 0.1 mM PIH medium. Rifabutin was tested at 0.25 mg/L with or without the specified concentration of a polymyxin agent. Samples were taken at 0, 2, 4, 8 and 24 hours in order to determine the colony forming units (cfu). Bactericidal activity is defined as ≥3-log reduction in cfu compared to the cfu present in the inoculum (0 hours). Synergy of the tested combination is defined as ≥2-log reduction in cfu compared to the cfu of the most active agent of the combination.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1-28. (canceled)

29. A method of treating an A. baumannii infection in a subject, the method comprising administering to the subject a dose of rifabutin and a subtherapeutic dose of a polymyxin.

30. The method of claim 29, wherein the dose of rifabutin is between about 400 mg/day and 600 mg/day.

31. The method of claim 29, wherein the dose of rifabutin about 400 mg/day.

32. The method of claim 29, wherein the dose of rifabutin is about 600 mg/day.

33. The method of claim 29, wherein the subtherapeutic dose of the polymyxin is a dose at which a mean fCₘₐₓ, fAUC and fC_{trough} required for clinical efficacy if given alone would not be achieved.

34. The method of claim 29, wherein the subtherapeutic dose of the polymyxin results in an fC_{trough} inhibiting growth of about 5% to about 50% of the strains in a test panel of 100 or more recent A. baumannii clinical isolates.

35. The method of claim 29, wherein the rifabutin and the polymyxin are administered in a single formulation.

36. The method of claim 29, wherein the rifabutin and the polymyxin are administered separately.

37. The method of claim 29, wherein the rifabutin and/or the polymyxin are administered intravenously.

38. The method of claim 29, wherein the A. baumannii infection is a carbapenem-resistant A. baumannii (CRAB) infection.

39. The method of claim 29, wherein the dose of rifabutin and the polymyxin act synergistically to inhibit the growth of A. baumannii.

40. The method of claim 29, wherein the polymyxin is polymyxin B.

41. The method of claim 40, wherein the subtherapeutic dose of polymyxin B is less than 1.5 mg/kg/day.

42. The method of claim 40, wherein the rifabutin and the polymyxin B are administered in a single formulation.

43. The method of claim 40, wherein the rifabutin and the polymyxin B are administered separately.

44. The method of claim 40, wherein the rifabutin and/or the polymyxin B are administered intravenously.

45. The method of claim 40, wherein the A. baumannii infection is a carbapenem-resistant A. baumannii (CRAB) infection.

46. The method of 40, wherein the dose of rifabutin and the polymyxin B act synergistically to inhibit the growth of A. baumannii.
